SEQUENCE LISTING (12 sequences)

SEQ ID NO: 1 - DNA, 22 bp, Artificial
  chemically synthesized
  misc_feature (1)..(22): forward primer for microsatellite marker ETH3
gaacctgcctctcctgcattgg

SEQ ID NO: 2 - DNA, 22 bp, Artificial
  chemically synthesized
  misc_feature (1)..(22): reverse primer for microsatellite marker ETH3
actctgcctgtggccaagtagg

SEQ ID NO: 3 - DNA, 22 bp, Artificial
  chemically synthesized
  misc_feature (1)..(22): forward primer for microsatellite marker ETH225
gatcaccttgccactatttcct

SEQ ID NO: 4 - DNA, 21 bp, Artificial
  chemically synthesized
  misc_feature (1)..(21): reverse primer for microsatellite marker ETH225
acatgacagccagctgctact

SEQ ID NO: 5 - DNA, 21 bp, Artificial
  chemically synthesized
  misc_feature (1)..(21): forward primer for microsatellite marker BM1824
gagcaaggtgtttttccaatc

SEQ ID NO: 6 - DNA, 21 bp, Artificial
  chemically synthesized
  misc_feature (1)..(21): reverse primer for microsatellite marker BM1824
cattctccaactgcttccttg

SEQ ID NO: 7 - DNA, 25 bp, Artificial
  chemically synthesized
  misc_feature (1)..(25): forward primer for microsatellite marker TGLA227
cgaattccaaatctgttaatttgct

SEQ ID NO: 8 - DNA, 24 bp, Artificial
  chemically synthesized
  misc_feature (1)..(24): reverse primer for microsatellite marker TGLA227
acagacagaaactcaatgaaagca

SEQ ID NO: 9 - DNA, 21 bp, Artificial
  chemically synthesized
  misc_feature (1)..(21): forward primer for microsatellite marker BM2113
gctgccttctaccaaataccc

SEQ ID NO: 10 - DNA, 21 bp, Artificial
  chemically synthesized
  misc_feature (1)..(21): reverse primer for microsatellite marker BM2113
cttcctgagagaagcaacacc

SEQ ID NO: 11 - DNA, 25 bp, Artificial
  chemically synthesized
  misc_feature (1)..(25): forward primer for cBM1824
atcagaatggactcagatttctcaa

SEQ ID NO: 12 - DNA, 22 bp, Artificial
  chemically synthesized
  misc_feature (1)..(22): reverse primer for cBM1824
attctccaactgcttccttgaa

(19) United States
(12) Patent Application Publication
(10) Pub. No.: US 2008/0085522 A1
(43) Pub. Date: Apr. 10, 2008

(54) Meat analysis technique

(75) Inventors: Ciaran Meghen, Co. Dublin (IE); Ronan Loftus, Dublin (IE); Carol Scott, Dublin (IE)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Parlanca Limited, Dublin (IE)

(21) Appl. No.: 11/973,275

(22) Filed: Oct. 5, 2007

(30) Foreign Application Priority Data
Oct. 10, 2006 (GB) 0620034.9

(51) Int. Cl. C12Q 1/68; C12Q 1/02

(52) U.S. Cl. 435/6; 435/29

(57) ABSTRACT

A method of determining the source of a component part of a multi-source comminuted meat product. The method comprising the steps of providing a sample of said meat product, isolating from said meat product a meat component substantially derived from a single source animal, analysing said isolated meat component to identify at least one traceable marker of the source animal.

The present invention relates to a method which is suitable for analysing a sample of a meat product to determine the origin of its constituent parts. It is particularly suited to analysing a sample of a multi-source comminuted meat product to determine the source of a batch of meat or an individual animal that has contributed to the meat product.

In an industrial environment minced (ground) beef is produced from meat from a number of different animals. A survey by researchers at Colorado State University found that “the smallest number of cattle contributing muscles and/or fat to a single 4-ounce ground beef patty was on average, 55 and the greatest number, on average, was 1,082” (unpublished). These animals typically originate from different farms, and possibly different slaughter plants or even countries. Retailers and catering outlets often purchase meat products from a range of different suppliers which further broadens the potential source of animals contributing to a product. In the event of a food safety issue product recall may be necessary and at a minimum stakeholders are required to effectively contain and manage the extent of any particular outbreak. Within the meat industry such recalls most frequently occur in the event of contamination of ground meat with microbes such as E. coli 0157, thought there are many other pathogens and other causes of such recalls. The situation is similar in other multi-source comminuted meat products. Examples of multi-source comminuted meat products include minced (ground) meat of all species, burgers, sausages, reformed ham, meat balls and meat loaf, though there are many more.

As a result of the diversity of source animals that potentially contribute to such meat products, a challenge for the industry has been how to effectively determine the source of meat contributing to a particular batch or end product. Effective traceability is seen as a highly desirable tool in risk assessment and management strategies, whilst also representing an important tool in maintaining public confidence in the integrity of the minced (ground) meat and other multi-source comminuted meat product chain.

Conventional approaches towards meat traceability require source meat to be processed in batches and the integrity of these batch structures be maintained throughout the supply chain. Such systems, if implemented robustly can lead to production inefficiencies or are challenging to maintain batch integrity. Furthermore, they only permit limited traceability as it may only be possible to trace a particular batch of ground meat to a large number of sources. For example, one of the larger North American meat plants slaughters up to 6,000 head per day, containing animals originating from many different farms. In a ground beef production batch meat trimmings could be mixed from different slaughter dates and different slaughter plants to yield batch sizes of >50,000 potential source animals. Such a problem of identification is well beyond conventional tagging or manual tracking technologies.

Calvo et al. disclose methods in which PCR techniques are used to identify beef contamination in pork products (J. Agric. Food Chem., SO (19), 5265-5261) and vice versa (J. Agric. Food Chem., SO (19), 5262-5264). However, these methods are restricted to identifying the presence of an abhorrent species of meat, but provides no indication of the individual source of meat within the meat product. Janssen et al. provide a method of using PCR to identify particular species of meat within a mixture of meats (Biomedical and Life Science, Vol. 21, No 3, Sept. 1998, 115-120), but again, no details regarding particular source animals is obtainable through this method.

There is thus a need in the area of meat production, processing and handling, particularly in the field of multi-source comminuted meat products, for improved techniques to allow tracing of the constituent parts of a meat product back to the source batch, or even the source animal or farm. Such techniques could also be used by stakeholders within a supply chain to, for example:
Identify or confirm the origins of a particular meat product (e.g. ground beef) to the production batch, even indicating contributing animals and/or farms.
Look for contaminating meat introduced through accident, adulteration or fraud, e.g. where a stakeholder further along the meat chain mixes meat from an non-approved source with a particular meat batch. Adulteration is a significant problem for many trade purchasers who cannot currently verify the composition of a quality product they are purchasing.
Indicate whether a particular batch meat originated from a particular source or not, i.e. if a problem arises, determining where the problem originated.
Provide an indication, e.g. a probability, of whether a particular suspect animal found its way into a particular batch meat.

According to the present invention there is provided a method of determining the source of a component part of a multi-source comminuted meat product, the method comprising the steps of:
(a) providing a sample of said meat product;
(b) isolating from said meat product a meat component substantially derived from a single source animal; and
(c) analysing said isolated meat component to identify at least one traceable marker of the source animal.

A multi-source comminuted meat product is a product which is formed from ground/minced, chopped, flaked or otherwise processed meat from several sources which is reformed or combined into a single meat product. Such products are extremely difficult or impossible to analyse using prior art techniques due to the mixture of constituent parts present. Some examples of multi-source comminuted meat products include burgers, sausages, mice/ground meat, reformed ham, meat balls and meat loaf, though there are many more. Preferred meat products for use in the present invention are minced/ground meat products and products derived therefrom, e.g. burgers and sausages.

The present invention is suitable for analysing meat products from any species of meat. Mention may be made of beef, lamb/mutton and pork, these generally making up the bulk of meat production, but the method is, of course, equally suited to other species such as venison, chicken, turkey, goat, horse etc.

In general the meat component derived from a single source animal is a single muscle fibre or single piece of fat tissue, or a collection of such fibres or pieces of tissue. Muscle tissue is generally formed as bundles of fibres, each containing many muscle cells. These can be identified within the meat product and isolated. As such fibres or bundles of fibres are derived from a single animal, they are suitable for use in the present method. Fat tissue is formed in a less ordered structure yet, after the mincing /grinding process, pieces of fat tissue retain structural integrity such that pieces of fat derived from a single source animal can be identified and isolated. Within such pieces of fat tissue smaller sub-units of fat tissue, which may be thread-like, can be identified and isolated. It is generally preferred that a single muscle fibre or piece of fat is used. Such a meat component may be obtained by dissection of the meat product, e.g. under magnification under a light microscope of magnifying glass. Alternatively, a smaller derivative could be used as the meat component could be used, e.g. a number of muscle or adipose cells.

Accordingly, step (b) may comprise isolating a single muscle fibre or piece of fat tissue from the meat source, which may optionally be achieved by manual dissection. Manual dissection is optionally be carried out under magnification, though it is achievable without the aid of magnification. The dissection may suitably involve removing a sample of fat and/or muscle from the meat source, e.g. with tweezers, which is then observed under a microscope. Such a sample of fat or muscle is typically about the size of a grain of rice for fat tissue and a short thin thread for muscle fibres. From the sample of fat or muscle a single muscle fibre or piece of fat tissue can then be removed, e.g. with tweezers. It is highly preferable that the meat component so obtained is not contaminated with other material from the meat source.

The meat component is preferably washed in a suitable liquid to remove or reduce any contamination on its surface, e.g. by agitation in double distilled water (ddH₂O) or suitable buffer. This washing step may help decrease contamination from traces of material from another source which may tend to adhere to the surface of the fibre. The washing step may include mechanical agitation.

It may be preferred that a fat sample is used in the present method. This is because it is generally quicker and easier to obtain a fat sample from the meat source than a muscle sample, and there is less potential for unobserved contamination of a fat sample than a muscle sample due to its colour. However, there may be situations where a meat fibre is preferred, e.g. in very lean meat products.

In general, it has been impossible in the prior art to conduct analysis of comminuted meat products due to the large mixtures of animals involved which are, by their nature, thoroughly mixed together. However, the present invention overcomes this difficulty by providing means through which it is possible to identify meat components in a comminuted meat product which are derived from a single source animal.

Step (c) may comprise analysing the meat component for a marker which is capable of correlating the meat component to a source animal or to a source batch. Typically the analysis is an analysis of genetic material. Analysing genetic material is extremely powerful in its ability to uniquely identify an individual animal from within a large population using a very small sample. Suitable genetic material may be DNA (genomic or mitochondrial) or RNA. In one preferred embodiment the analysis is based on analysis of genomic DNA, especially via the polymerase chain reaction (PCR). Alternatively, though less preferably, the analysis could be based on mitochondrial DNA.

Accordingly the method may comprise the step of extracting genetic material, preferably DNA, from the meat component for further analysis.

In a preferred embodiment the analysis may be based on single nucleotide polymorphism (SNP) profiling. SNP analyses focus on single point mutations helping build a unique individual profile when repeated over multiple SNPs. SNPs can be detected by various techniques known in the art (for example Exemplary SNP-bases techniques are set out in “Selection and use of SNP markers for animal identification and paternity analysis in U.S. beef cattle” Journal Mammalian Genome—Volume 13, Number 5/May, 2002, Pages 272-281 Michael P. Heaton, Gregory P. Harhay, Gary L. Bennett, Roger T. Stone, W. Michael Grosse, Eduardo Casas, John W. Keele, Timothy P. L. Smith, Carol G. Chitko-McKown, William W. Laegreid). Restriction fragment length polymorphism (RFLP analysis is a conventional technique for SNP detection. However, RLFP analysis is generally labour intensive and relies on the presence of relatively large quantities of genetic material for analysis; a large quantity of genetic material may, however, be provided using whole genome amplification which is discussed later. A particularly preferred technique is the use of fluorescence resonance energy transfer (FRET) based techniques. FRET, when combined with PCR based techniques provides a convenient and cost effective way to carry out SNP profiling. Such techniques are known in the art, and analysis kits are available from, for example, KBiosciences Hoddesdon, UK. Alternative techniques which may also be preferred, primarily on the basis of their speed and convenience relative to RFLP analysis, are sequencing-based, PCR-based, microarray-based, or high performance liquid chromatography (HPLC)-based techniques. Microarray technology has potential advantages in terms of rapid, simple and highly convenient analysis. Accordingly, the method may suitably comprise performing SNP profiling on the meat component. Any suitable SNP's for the meat species being investigated may be used. Suitable SNP's for use with beef are provided at http://cgemm.louisville.edu/usmarc/servlets/PrintData?FILE NAME=SNP_summary, and other lists of SNPs for other animals publically available.

In another embodiment, the analysis may be based on microsatellite (short tandem repeat—STR) profiling—analogous to DNA fingerprinting. In STR analysis, variations in the length of microsatellites are used to provide a unique profile of an individual animal. The technique is generally achieved using PCR which means it can be used on very small quantities of genetic material. Accordingly, the method may suitably comprise performing DNA microsatellite profiling on the meat component, preferably PCR-based microsatellite profiling.

Further details of genetic analysis in relation to meat identification is to be found in Cunningham and Meghan, Rev. sci. tech. Off. int. Epiz., 2001, 20 (2), 491-499.

Alternative forms of genetic testing are well know in the art and may be used, and such suitable techniques would be apparent to the person skilled in the art. Such techniques include, but are not limited to, restriction fragment length polymorphisms (RFLPs) amplified fragment length polymorphism (ampFLP) or other PCR based techniques.

In certain embodiments the present invention therefore combines the ability of DNA to uniquely identify animals with the requirement for only forensic amounts of source material to uniquely identify individuals, to provide a robust means of tracing the constituent parts of a particular meat product.

In general it is extremely useful if the results of the analysis of step (c) are suitable for comparison with previously obtained data relating to animals which have been slaughtered to allow identification of the source of the meat component. Accordingly, the analysis used would generally be selected to provide results corresponding to stored data relating to the source animals. It is envisaged that databases of stored information relating to markers of particular slaughtered animals will be developed to aid the traceability of the origin of meat. It is likely that such databases will store a DNA profile for each animal slaughtered. The type of profile used may vary, but it is likely that the data will be based on SNP or microsatellite profiling as this is a simple, cheap and highly automatable technique suitable for providing highly specific and readily resolvable profiles of individual animals. The number and identity of specific SNPs or microsatellites profiled is, of course, important in this situation, and thus the method of the present invention would typically use corresponding microsatellites to those used to generate such database.

The method may accordingly further comprise comparing the at least one traceable marker of the source animal, with a database of corresponding markers of potential source animals. In a preferred embodiment the method may comprise comparing the genetic profile (e.g. microsatellite profile) of genetic material extracted from the meat component, with a database of corresponding genetic profiles of potential source animals. Through this step it is possible to determine the source of the component parts of a meat product. This has utility in a number of ways, e.g. identifying the source of contaminated meat or identifying meat which has been added fraudulently. This provides a powerful tool for an interested party to investigate the provenance of a meat product.

In some circumstances it may be desirable to amplify the DNA obtained from the DNA extraction step prior to performing DNA analysis. This may conveniently be achieved by whole genome amplification (WGA). Suitable techniques to achieve this will be apparent to the person skilled in the art, and kits to achieve WGA are commercially available from Sigma under the trade name GenomePlex (e.g. WGA-1 to WGA-4), or from Qiagen under the trade name REPLI-g. The Sigma technology is based on fragmentation of the genome into a library of short overlapping fragments (˜400 bp) which is primed and replicated by limited PCR. The Qiagen technology is based on multiple displacement amplification (MDA), a non-PCR based technology, using a unique highly processive DNA polymerase and randomised primers to replicate the genome by isothermal amplification. Other WGA systems could of course be used (other methods of WGA are discussed in Barker et al. Genome Research 14:901-907, 2004). The advantage of performing a WGA step is that the content of DNA may be increased in a non-specific manner prior to analysis; this may be particularly useful if very small quantities of DNA are obtained in the extraction step, i.e. below those levels required for the particular analytical technique selected. WGA can be achieved without adversely affecting the result of the subsequent analysis, e.g. SNP analysis.

The method may suitably comprise analysing samples from more than one meat component obtained from the meat product. By analysing a plurality of meat components from a meat product it is possible to build up an representation of the various sources from which the meat product is constituted. Typically the method of the present invention involves taking 10 or more, preferably 20, 30, 40 or 50 or more meat components and analysing them. The more meat components which are analysed, the more complete the picture of the various origins of the meat product will become. However, there is also an increase in the chance of obtaining duplicate (i.e. redundant) meat components. When a large number of samples are taken it may also be possible to build up a picture of the proportion of meat from various sources which make up the meat product. It should, however, be noted that, in many cases, the method of the present invention need not reveal each and every source of meat in a meat product. In some embodiments, obtaining details of the origin of a reasonable number of source animals (e.g. from 5 to 50) would provide, in combination with production records, a fair indication of the source of much of the meat product, i.e. the key contributory abattoirs, forum, processing plants or production batches. If such analysis was performed over a number of occasions (e.g. following repeated occurrence of a pathogen) a more detailed picture could be established by virtue of the repeated appearance of a particular source, e.g. a particular farm, abattoir or processing plant. In other embodiments the detection of a single abhorrent animal may be suitable to determine that the meat product does not meet particular required criteria for the meat product, for example meat from an animal slaughtered in a non-kosher way present in a meat product which is claimed to be kosher, or meat from non-specification animals introduced inadvertently or fraudulently.

The method of the present invention may also involve comparing the results of analysis of a number of meat components with a database and determining whether a particular animal, or an animal, or an animal from a particular source (e.g. from an abattoir) is contained within the meat product. In certain embodiments a probability of such an animal being present in a particular meat product may be obtained.

The method may comprise the step of identifying a meat component which contains meat derived from more than one source and eliminating it from further consideration. It is possible that a meat component isolated in the method of the present invention will contain meat from more than one animal despite efforts to minimise this. In such an occurrence the contaminated meat component will generally be identified and disregarded. Where genetic analysis is carried out it is relatively simple to identify samples where contamination has occurred, i.e. by visualising multiple profiles. In the case of SNP profiling, the presence of multiple animals is manifest through excess heterozygosity and the lack of repeatability of results.

It may be preferable that the analysis of a meat component is repeated to reduce the possibilities of erroneous results and to detect allelic dropout. For example, repetition may reduce errors associated with PCR processes, such as allelic drop-out.

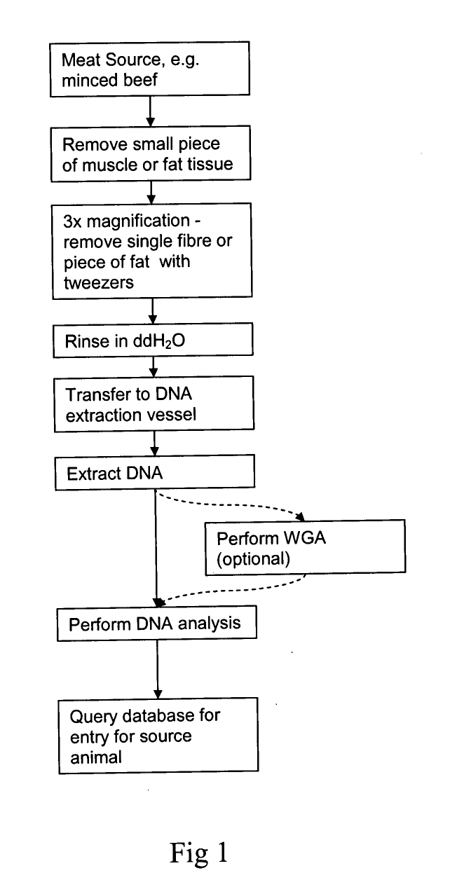

The present invention will now be further described, by way of example only, with reference to the accompanying drawing (FIG. 1) which is a flow chart illustrating an embodiment of the method of the present invention.

### EXAMPLE 1

### Procedure for Obtaining a Meat Fibre from a Meat Product

The following method is followed to obtain a fibre from a comminuted meat product, the fibre being unique to a single animal which contributes to the meat product. The procedure described relates to a method of obtaining a sample of fat from minced (ground) meat. However, the process could be simply modified to obtain a fibre of muscle, and the meat product could of course be other comminuted meat products.

TABLE 1
Materials Used

| Reagents | Consumables | Equipment |
|---|---|---|
| ddH₂O | 96 well extraction plates/extraction tubes | Light microscope |
| 70% | Plastic toothpicks | Tweezers |
| Ethanol | Petri dishes | |
| | Plastic dropper | |

Procedure
1. Select the sample of ground meat.
2. It is preferable to work on fresh/defrosted mince rather than a frozen meat.
3. Take a sample from the ground meat. This is further subdivided by taking a small piece of fat from the mince product using a plastic toothpick and/or clean tweezers and place in a Petri dish. It is important to take an individual piece of fat (about the size of a grain of rice), ideally with no visible meat attached to it.
4. Place the Petri dish under the microscope set at a suitable magnification, e.g. (3X).
5. Examine the fat sample and tease out a single fat thread using a plastic toothpick and/or clean tweezers. Ensure no contaminating material is visibly attached to the thread.
6. Clean tweezers between manipulations using 70% ethanol and wiping with tissue—as an additional measure the tweezers may be passed over a flame to destroy any contaminating tissue.
7. Place a small drop of ddH₂O (˜500 μl) in a Petri dish using a plastic dropper. Using a new plastic toothpick, transfer the thread to the drop of ddH₂O.
8. Using the toothpick agitate the thread in the water for 1-2 minutes.
9. Transfer the thread to the designated well in the extraction plate or extraction tube using the toothpick
10. Check that transfer has been successful by examining the toothpick visually (optionally under the microscope) to ensure the thread has been removed. It may also be possible to view the sample in the well.

This technique is relatively fast, allowing over 35 samples to be taken in a 45-60 minute period.

### EXAMPLE 2

### Trial of Method in Packaged Meat

In order to test the process set out in Example 1 and the efficacy of the process overall, the procedure was tested on a large scale.

5 packs of a mince product produced in a commercial meat processing plant were obtained. The mince was produced during the first production shift to minimise cross-over between production batches. The 5 mince subsamples were collected at different stages of the mincing process (1 start of batch, 2-4 from the middle of the batch and the 5^{th} at the end). The muscle trim and primals used in the mince batch were determined to have come from 77 carcases constituting a single mince production batch in the abattoir—this was determined using the abattoir production records. In addition, each of the contributing carcasses had previously been DNA sampled so their DNA profiles could readily be generated.

The microsatellite DNA profiles for the input animals were obtained using conventional PCR techniques. The primers flanking the STRs are shown in table 2.

TABLE 2
Primers for STR analysis.

| | | | | | |
|---|---|---|---|---|---|
| | | | Anneal | | |
| | | | temp. | | |
| No. | Primer | Range (bp) | ° C. | Sequence 5′ - 3′ | FOR/REV |

| | | | | | |
|---|---|---|---|---|---|
| 4 | ETH3 | 117-129 | 58 | GAACCTGCCTCTCCTGCATTGG | FOR |
| | | | | ACTCTGCCTGTGGCCAAGTAGG | REV |
| 5 | ETH225 | 140-156 | 58 | GATCACCTTGCCACTATTTCCT | FOR |
| | | | | ACATGACAGCCAGCTGCTACT | REV |
| 6 | BM1824 | 178-190 | 58 | GAGCAAGGTGTTTTTCCAATC | FOR |
| | | | | CATTCTCCAACTGCTTCCTTG | REV |
| 7 | TGLA227 | 78-104 | 58 | CGAATTCCAAATCTGTTAATTTGCT | FOR |
| | | | | ACAGACAGAAACTCAATGAAAGCA | REV |
| 1 | BM2113 | 125-143 | 55 | GCTGCCTTCTACCAAATACCC | FOR |
| | | | | CTTCCTGAGAGAAGCAACACC | REV |
| 11 | cBM1824 | 231-243 | 58 | ATCAGAATGGACTCAGATTTCTCAA | FOR |
| | | | | ATTCTCCAACTGCTTCCTTGAA | REV |

Samples were taken from the 5 packs of mince products following the process of Example 1 were obtained. DNA was extracted by the Cresol Red extraction (details of this extraction technique are provided in WO/98/39475 and are not repeated here) and microsatellite DNA profiles were determined using conventional techniques.

Results

A total of 90 mince extractions were carried out. (3 sets, each set consisted of 30 samples taken from the 5 mince packs). The results are summarised in Table 3.

TABLE 3
Extraction Results

| | Mince | Mince | Mince | |
|---|---|---|---|---|
| Profiles | extract 1 | extract 2 | extract 3 | Total |
| Single | 18/30 | 13/30 | 25/30 | 56/90 |
| | (60%) | (43%) | (83%) | (62%) |
| Mixed | 3/30 | 6/30 | 5/30 | 14/90 |
| (more than 1 DNA | (10%) | (20%) | (17%) | (16%) |
| profile) | | | | |
| Partial/Weak | 6/30 | 5/30 | — | 11/90 |
| (unreadable) | (20%) | (17%) | | (12%) |
| No Result | 3/30 | 6/30 | — | 9/90 |
| | (10%) | (30%) | | (10%) |

Overall the percentage of single, clean DNA profiles is fairly high (62%), with the rest of the samples either producing no result or mixed (i.e. DNA from multiple animals). This indicates that the process of Example 1 for the removal of meat components from a single source animal from the mince product works well. The process could perhaps be further optimised, e.g. by increasing the stringency of the rinsing step.

The “single” DNA profiles were checked against the DNA profiles for the 77 input animals. For all 3 sets of extractions, many (but not all) of the profiles could be matched to an input animal. A number of the “single” profiles were unique and could not be matched to any of the input animals. The results are summarised in Table 4.

TABLE 4
Single Profile Summary Table

| | Mince | Mince | Mince | |
|---|---|---|---|---|
| | extract 1 | extract 2 | extract 3 | |
| | (Trial 11) | (Trial 13) | (Trial 15) | Total |
| Total Single | 18/30 | 13/30 | 25/30 | 56/90 |
| Profiles | (60%) | (43%) | (83%) | (62%) |
| Match to | 10/30 | 4/30 | 5/30 | 19/90 |
| input DNA | (33%) | (13%) | (17%) | (21%) |
| profile | (6 different | (3 different | (2 different | (9 different |
| | profiles) | profiles) | profiles) | profiles) |
| No match to | 8/30 | 9/30 | 20/30 | 37/90 |
| input DNA | (27%) | (30%) | (66%) | (41%) |
| profile | (6 different | (6 different | (11 different | (16 different |
| | profiles) | profiles) | profiles) | profiles) |

Overall 21% of the 90 mince extractions could be matched to 9 of the input animals which, in combination with plant production records, facilitated the identification of the production batch of origin of the mince.
41% of the 90 mince extractions produced apparently single DNA profiles but these profiles did not match any of the input animals (16 different profiles).
To ensure the unidentified DNA profiles were not the result of experimental error or a PCR anomaly the extractions were repeated, and the samples reanalysed. This generated the same unidentifiable profiles.
Furthermore, the same unidentified DNA profiles appeared in different extraction sets.
The unidentified DNA profiles appear to be genuine DNA profiles, most likely from animal material not considered to have contributed to the mince product. Thus, the method of the present invention has demonstrated its ability to detect traceability errors in the mince production process and ultimately to identify the presence of meat originating from animals which should not be present in the production batch.

### EXAMPLE 3

### Confirmation of Replicability in US Ground Beef

The following experiment was performed to ensure the general process was suitable for use in ground meat generated under different processing conditions. The mince used in the present example was sourced in the US, whereas that used in Example 2 was sourced in Ireland. It is useful to determine if the system is robust across different types of mince.

A pack of US ground beef and a pack of US ground chuck were obtained for testing. A number of samples were removed following the method of Example 1 and analysed in the same way as the Irish mince product.

The results were very similar to the results for the trials on the Irish mince product with 60% single profiles (12 unique DNA profiles).

This suggests that the developed method and results for the previous trials are applicable to ground beef products generated under a variety of processing conditions, such as those typically encountered in North America. Furthermore, the results indicate the robust nature of the technique of the present invention.

### EXAMPLE 4

### Use of Whole Genome Amplification (WGA)

As a consequence of relying on forensic sized samples for analysis the single fibre samples may not always contain sufficient quantities of DNA for automated DNA analyses involving multiple SNPs or STRs.

The present experiment was performed to determine whether whole genome amplification (WGA) technology would be of benefit in amplifying total DNA before DNA profiling of the samples. WGA has already been demonstrated as a means of amplifying total DNA from relatively small amounts of starting material, yielding microgram quantities of DNA. (see for example http://www.sigmaaldrich.com/sigma/general% 20information/wga_applications_poster.pdf#search=%22allelic%20dropout %20snp%22)

There are currently a variety of methods used by laboratories carrying out WGA. For this experiment the Omniplex® technology developed by Sigma was used, more specifically a 10 reaction kit (WGA-2). The OMNIPLEX® method involves chemical fragmentation of the genome into a library of short overlapping fragments (˜400 base pairs) which is primed and replicated by limited PCR.

Five mince samples were extracted using the Cresol red extraction method discussed previously. The DNA concentration of samples derived from the extraction was determined by a fluorometer assay. All the mince extractions had low initial concentrations of DNA. They were then amplified using the WGA process according to the manufacturers instructions.

The WGA products were again quantified using the fluorometer assay. Overall, there was significant amplification of the DNA by the WGA kit, producing DNA of sufficient quantity for downstream applications.

TABLE 4
Summary of WGA amplification.

| | Initial [DNA] | WGA [DNA] | Amplification |
|---|---|---|---|
| Sample | ng/μL | ng/μL | (fold) |
| Sigma 1 | 0.415 | 53.85 | 130 |
| Sigma 2 | 7.116 | 117.62 | 17 |
| Sigma 3 | 0.809 | ˜50 | 60 |
| Sigma 6 | 135.4 | 89.41 | — |

The original mince extracts and the WGA products underwent PCR as described previously to determine the microsatellite DNA profiles and to examine whether the WGA process lead to any differences between the DNA profiles generated before and after WGA, i.e. whether any PCR anomalies such as allelic dropout had occurred. The results indicated that the WGA worked extremely well with all samples producing strong, clean, single profiles which were the same profile as the DNA extract prior to WGA.

The results of this protocol demonstrate that WGA may be a useful technique where very small quantities of DNA must be analysed.

1. A method of determining the source of a component part of a multi-source comminuted meat product, the method comprising the steps of:
(a) providing a sample of said meat product;
(b) isolating from said meat product a meat component substantially derived from a single source animal; and
(c) analysing said isolated meat component to identify at least one traceable marker of the source animal.

2. The method of claim 1 wherein the meat product is minced or ground meat or a product derived therefrom.

3. The method of claim 1 wherein step (b) comprises isolating a single muscle fibre or piece of fat tissue from the meat source.

4. The method of claim 3 wherein step (b) includes manual dissection of the meat product.

5. The method of claim 1 wherein the meat component so obtained is not contaminated with other material from the meat source.

6. The method of claim 5 further comprising the step of washing the meat component in a suitable liquid to remove or reduce any contamination on its surface.

7. The method of claim 1 wherein step (c) comprises analysing the meat component for a marker which is capable of correlating the meat component to a source animal or to a source batch.

8. The method of claim 7 wherein step (c) comprises genetic analysis.

9. The method of claim 1 further comprising the step of extracting genetic material from the meat component.

10. The method of claim 9 wherein the genetic material is DNA.

11. The method of claim 9 comprising the step of amplifying the DNA obtained from the DNA extraction step prior to performing DNA analysis.

12. The method of claim 11 comprising conducting whole genome amplification of the DNA obtained from the DNA extraction step.

13. The method of claim 1 wherein the analysis of step (c) comprises single nucleotide polymorphism profiling.

14. The method of claim 13 wherein the single nucleotide polymorphism profiling comprises fluorescence resonance energy.

15. The method of claim 1 wherein the analysis of step (c) comprises microsatellite profiling.

16. The method of claim 15 wherein the microsatellite profiling comprises polymerase chain reaction.

17. The method of claim 1 further comprising comparing the at least one marker of the source animal or source batch, with a database of corresponding markers of potential source animals.

18. The method of claim 17 comprising comparing a genetic profile of genetic material extracted from the meat component, with a database of corresponding genetic profiles of potential source animals.

19. The method of claim 1 comprising analysing samples from more than one meat component obtained from the meat product.

20. The method of claim 19 wherein ten or more meat components are analysed.

21. The method of claim 1 comprising comparing the results of the analysis of a plurality of meat components obtained from a meat product with a database and determining whether a particular animal, or an animal from a particular source, is contained within the meat product.

22. The method of claim 1 comprising the step of identifying a meat component which contains meat derived from more than one source and eliminating it from further consideration.

23. The method of claim 22 wherein the meat component is identified through at least one of excess heterozygosity and the lack of repeatability of results.
